(19) United States
(12) Patent Application Publication
(10) Pub. No.: US 2016/0256638 A1
(43) Pub. Date: Sep. 8, 2016

(54) THERAPY GAS STORAGE AND DELIVERY APPARATUS

(71) Applicant: NEOGENIX, LLC, FT. LAUDERDALE, FL (US)

(72) Inventors: Srinivasan Sarangapani, Walpole, MA (US); Lawrence J. Cali, East Falmouth, MA (US)

(21) Appl. No.: 15/060,587

(22) Filed: Mar. 3, 2016

Related U.S. Application Data
(60) Provisional application No. 62/127,743, filed on Mar. 3, 2015.

(51) Int. Cl. A61M 13/00; A61M 39/22; A61F 13/00

(52) U.S. Cl. CPC A61M 13/003; A61F 13/00063; A61M 39/22; A61M 2202/0208; A61M 2202/0216; A61M 2202/0266; A61M 2202/0275; A61M 2205/502; A61M 2205/18

(57) ABSTRACT

A mobile dressing for delivering pressurized therapy gas to a wound. The dressing may include an inflatable structure for creating a sealed cavity around the wound. The inflatable structure further may include a dimple and an aperture within the dimple. A first lumen may be fluidly connected to the aperture for delivering pressurized therapy gas to the dimple. A second lumen may be fluidly connected to the opening for delivering pressurized gas to the inflatable structure. A first pump may include a first intake port fluidly connected to a first reservoir of therapy gas, as well as a first discharge port fluidly connected to the first lumen for supplying therapy gas at an increased pressure. A second pump may pressurize air for inflating the inflatable structure and creating a sealed cavity over the wound. Pressurized therapy gas may be administered to the wound in the sealed cavity.

### CROSS REFERENCE TO RELATED APPLICATIONS

This application claims the benefit of U.S. Provisional Application No. 62/127,743 filed on Mar. 3, 2015, the content of which is incorporated by reference herein in its entirety.

### FIELD OF THE INVENTION

The present invention generally relates to wound care. More particularly, this invention relates to storage and delivery devices for the application of a therapeutic gas or a mixture of gases to a wound.

### BACKGROUND

Therapeutic gases may be used to treat wounds or other conditions, including acute wounds, chronic wounds, pressure ulcers, and diabetic foot ulcers. For example, a dressing may be applied to a chronic wound to form an airtight seal, and a supply of therapy gas may be connected via a tube to the dressing to blanket the wound with the therapy gas. Transdermal oxygen therapy may accelerate wound healing by various mechanisms including: energizing ischemic cells to stimulate the natural healing process and promoting the production of collagen, granulation tissue, new blood vessels and skin. Ozone (O₃) therapy may be administered for the treatment of diabetic foot ulcers and other disorders. Nitric oxide (NO), carbon dioxide (CO), and hydrogen sulfide (H₂S) also may be useful for the topical treatment of wounds, conditions, or disorders. A need exists for new therapeutic gas storage and delivery devices and systems that may improve patient outcomes and expand access to patients with limited mobility or clinical support.

### SUMMARY

Hence, the present invention is directed to a mobile system for the application of transdermal oxygen, other therapeutic gases, or mixtures thereof to a wound. A mobile system for therapeutically delivering pressurized therapy gas to a wound may include an inflatable structure for creating a sealed cavity around the wound. The inflatable structure may include a flexible container for receiving pressurized gas. The flexible container may include an interior chamber for containing pressurized gas, and an opening in the interior chamber for receiving pressurized gas. The flexible container further may include a wound-facing surface which includes a dimple, and an aperture within the dimple. A first lumen may fluidly connect to the aperture for delivering pressurized therapy gas to the aperture, and a second lumen may fluidly connect to the opening for delivering pressurized gas to the interior chamber. A first pump may include a first intake port fluidly connected to a first reservoir of therapy gas at a first pressure, and a first discharge port fluidly connected to the first lumen for supplying therapy gas from the first reservoir at a second pressure that is greater than the first pressure. A second pump may include a second intake port fluidly connected to a second reservoir of gas at a third pressure, and a second discharge port fluidly connected to the second lumen for supplying pressurized gas from the second reservoir at a fourth pressure that is greater than the third pressure. The mobile system may include a plurality of operable configurations. In a first operable configuration, the interior chamber may contain a first volume of gas from the second reservoir at the third pressure, and in a second operable configuration, the interior chamber contains a second volume of gas from the second reservoir at the fourth pressure. In the second operable configuration, the first discharge port may supply therapy gas from the first reservoir to the aperture at the second pressure to treat the wound.

### DESCRIPTION OF THE DRAWINGS

In the accompanying drawings, which form a part of the specification and are to be read in conjunction therewith and in which like reference numerals (or designations) are used to indicate like parts in the various views:

FIG. 1 presents a block diagram of a method for therapeutically applying gas to a wound;

FIG. 2 is a schematic diagram of an embodiment of an apparatus for therapeutically applying gas to a wound in accordance with the method of FIG. 1;

FIG. 3 is a process flow diagram of an exemplary embodiment of a therapeutic gas storage and delivery system for use in therapeutically applying gas to a wound;

FIG. 4 presents an exemplary embodiment of a mobile dressing for therapeutically delivering pressurized gas to a wound;

FIG. 5 depicts another embodiment of a mobile device for storing and therapeutically delivering pressurized gas to a wound;

FIG. 6 presents a block diagram of another method for therapeutically applying gas to a wound;

FIG. 7 is a schematic diagram of an embodiment of an apparatus for therapeutically applying gas to a wound in accordance with the method of FIG. 6;

FIG. 8 is a schematic diagram of another embodiment of an apparatus for therapeutically applying gas to a wound in accordance with the method of FIG. 6;

FIG. 9 is a process flow diagram of another embodiment of a therapeutic gas storage and delivery system for use in therapeutically applying gas to a wound;

FIG. 10 is a process flow diagram of yet another embodiment of a therapeutic gas storage and delivery system for use in therapeutically applying gas to a wound;

FIG. 11 depicts another embodiment of a mobile device for storing and therapeutically delivering pressurized gas to a wound;

FIG. 12 depicts another embodiment of a mobile device for storing and therapeutically delivering pressurized gas to a wound;

FIG. 13 depicts another embodiment of a mobile device for storing and therapeutically delivering pressurized gas to a wound; and

FIG. 14 depicts another embodiment of a mobile device for storing and therapeutically delivering pressurized gas to a wound.

### DESCRIPTION

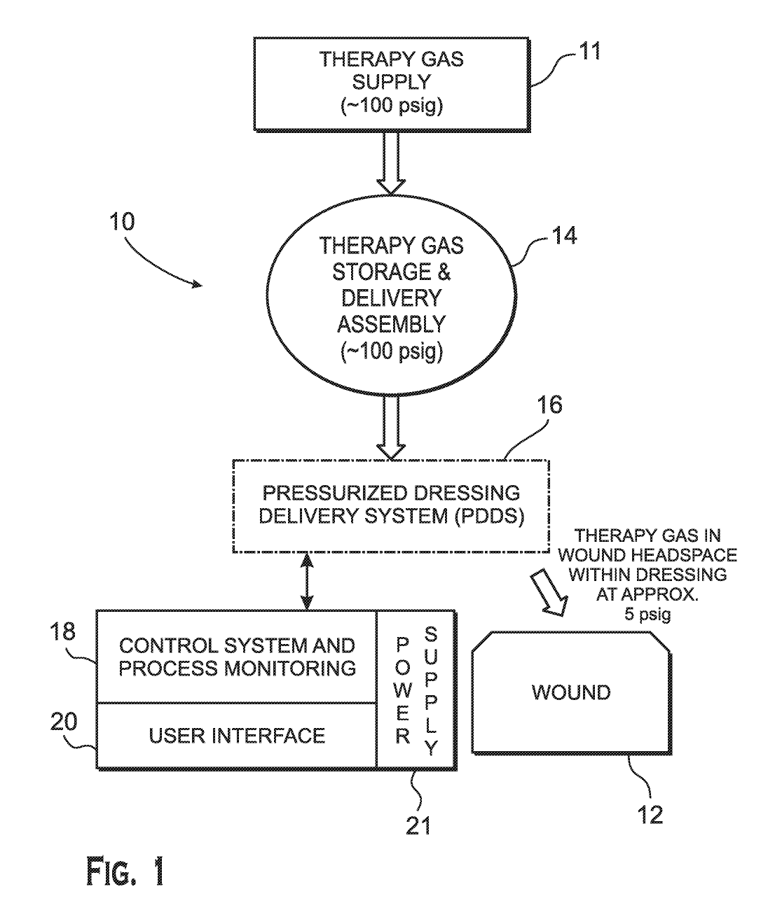

FIG. 1 presents a block diagram of an apparatus 10 for delivering therapeutic gas (therapy gas) to a wound 12. The apparatus 10 may include a gas storage and delivery device 14 for supplying the apparatus with therapy gas during wound care, a pressurized dressing 16 for delivering therapy gas to the wound 12 being treated, and a control system 18 which may include a user interface 20 for regulating operation of the apparatus. The control system 18 may include sensors which measure operating parameters of the apparatus. The control system 18 may include a microprocessor, memory, application specific integrated circuit(s) and/or a microcontroller, as well as other semiconductor devices and electronic components. The control system may include communication capabilities. The apparatus may include a power supply. Also, mechanical devices and non-electronic electrical circuit components may be employed to regulate operation of the apparatus. Generally, the apparatus 10 may be used to heal a wound by administering therapy gas, such as oxygen, ozone, chlorine dioxide, nitrogen, nitric oxide, medical grade air, or a mixture thereof to the wound bed.

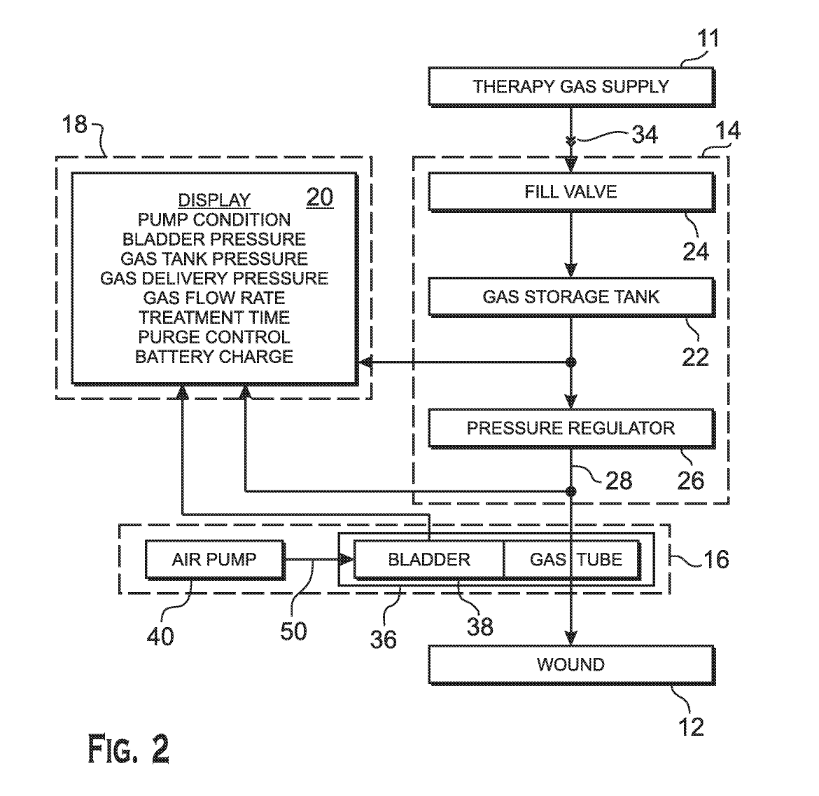

Referring to FIG. 2, the therapy gas storage and delivery device 14 may include a gas storage tank 22, fittings (e.g., fill valve 24 and pressure regulator 26), and flexible tubing 28 which supply the pressure dressing delivery system 16 with medical grade therapy gas. The tank 22 may be refillable and may be constructed of stainless steel or lined stainless steel, but other suitable configurations and materials for storing and dispensing compressed and/or liquefied gas may be used. The gas storage tank may be sized for portability and weight, as well as the desired delivery wound pressure and treatment duration. For example, the gas storage tank may be a pressure cylinder capable of supplying therapy gas continuously for 7-10 days at a service pressure of at least 5 psig. A gas storage and delivery device is disclosed in U.S. patent application Ser. No. 13/457,564, entitled “Oxygen Delivery Device,” filed on Apr. 27, 2012 (the '564 application). The '564 application is incorporated herein by reference in its entirety. Although, the therapy gas may be stored in the tank 22 at a pressure of approximately 100 psig, other suitable pressure levels may be selected to achieve clinical and operational objectives.

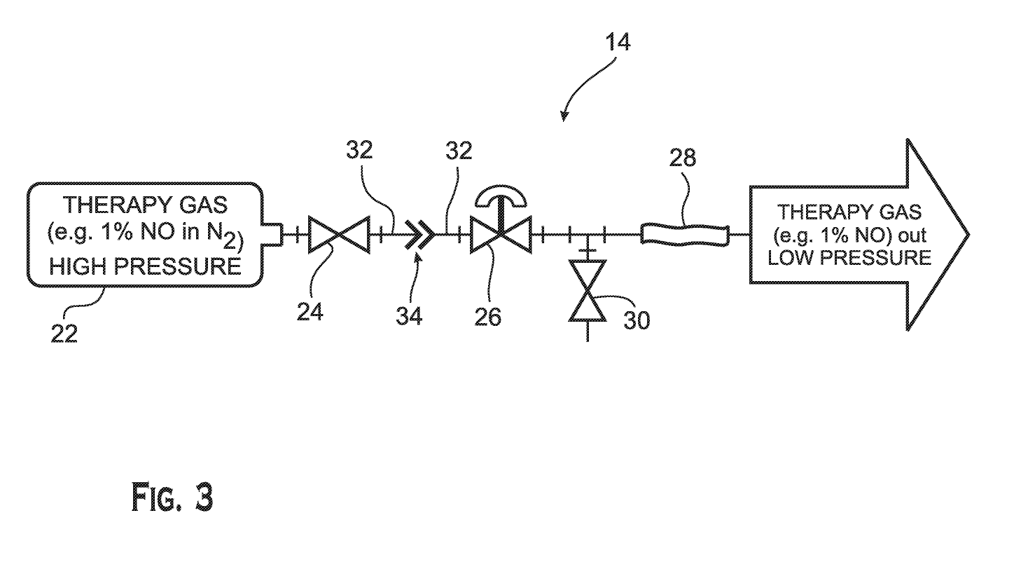

Referring to FIG. 3, a portable therapy gas storage and delivery assembly 14 may include a 10 cm³ capacity gas cylinder (e.g., Swagelok Part #53-4CS-TN-10) 22, a shut off valve (e.g., Swagelok Part #SS-41S2) 24, a gas regulator (e.g., Beswick Engineering Part #PRD2-1N1-O-6VK) 26, and a bleed valve (or purge valve) (e.g., Swagelok Part #SS-41S2) 30. The fittings on the high pressure side of the regulator may be connected by suitable metal pipe or flexible hose 32. Connection to these principal high pressure components may be made using compression fittings (e.g., Swagelok compression fittings) 34. By contrast, the fittings on the low pressure side of the regulator 26 may be connected by flexible tubing 28 made from a polymeric material suitable for use in hospital applications. Suitable materials for use in the low pressure tubing include, but are not limited to: silicone, polyethylene, polypropylene, polyurethane and various other thermoplastics.

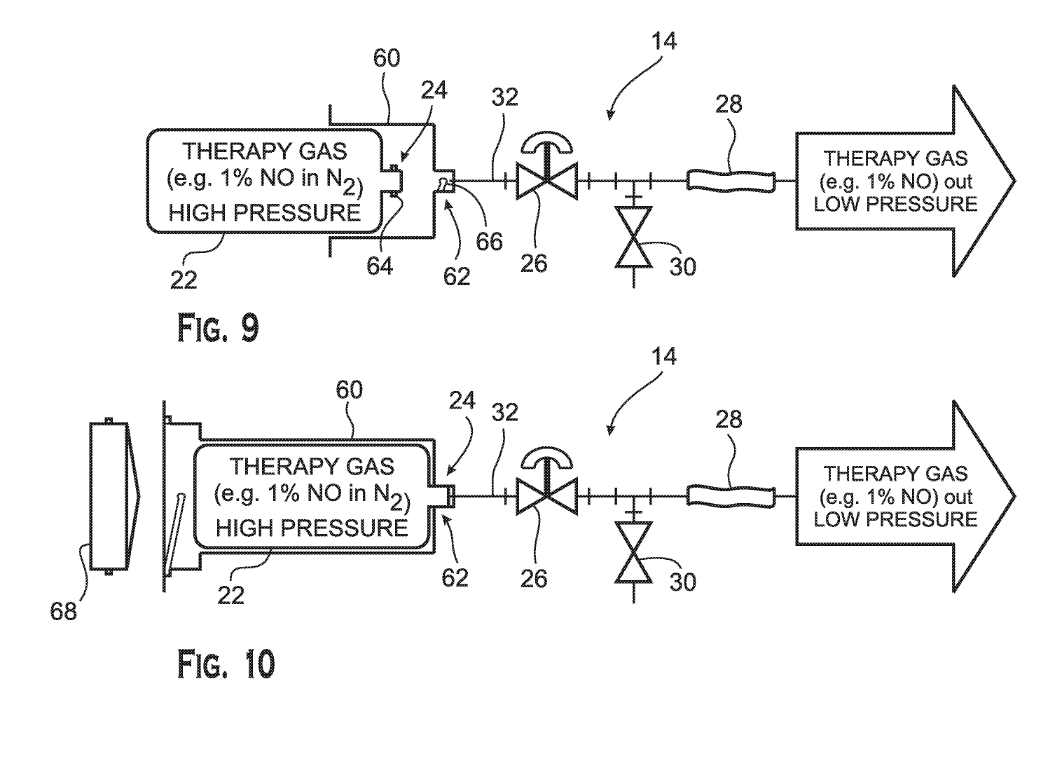

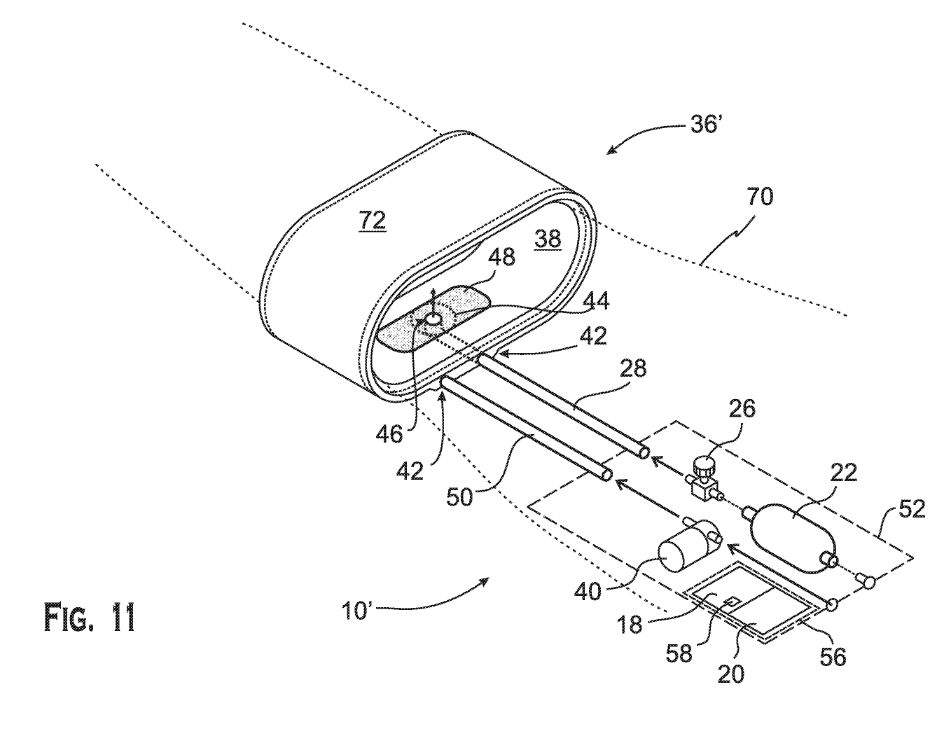

As shown in FIG. 9 and FIG. 10, the gas cylinder 22 may be connected to the fittings on the high pressure side of the regulator 26 by a quick connect fitting 62. In FIG. 9, the quick connect fitting 62 may be a bayonet fitting. For example, a male-bayonet fitting 64 may be disposed on the fill port of the cylinder. The male-bayonet fitting may be inserted into a female-bayonet fitting 66 that is fluidly connected to the high pressure fittings and components. Locking lugs on the male-bayonet fitting 64 may be aligned with engagement slots on the female-bayonet fitting 66. The locking lugs may be pushed into the engagement slots and twisted clockwise to lock the fitting in place. The female-bayonet fitting may be part of a housing which secures the cylinder into the portable therapy gas storage and delivery assembly 14. In FIG. 10, the quick connect fitting 62 may include a receptacle 60 that receives the cylinder. A cover 68 may mate with a portion of the receptacle 60 to lock the cylinder in the receptacle. For example, the cover 68 may be secured by a quarter turn bayonet fitting or screw threads. The gas cylinder 22 may be connected to the fittings on the high pressure side of the regulator by a pin or fitting in the receptacle that presses against and opens a gas valve 24 on the cylinder.

Additionally, the gas storage tank 22 may be disposable. The gas storage tank may be sized for portability and weight, the desired delivery pressure near the wound site, clinical needs and/or a prescribed treatment duration. For example, the gas storage tank may be a pressure cylinder capable of supplying therapy gas continuously for 7-10 days at a service pressure of at least 1.5 psig.

In use, the system 14 may be used to deliver therapy gas for wound treatment. The therapy gas may include oxygen, ozone, chlorine dioxide, nitrogen, nitric oxide, medical grade air, or a mixture thereof to the wound bed. For example, one therapy gas may be a gaseous mixture of 1% (by mass) nitric oxide (NO) and 99% (by mass) nitrogen (N₂). In this example, nitric oxide may be considered a therapeutic agent and nitrogen may be considered a carrier gas.

Referring to FIG. 3, a prototype of the therapeutic gas and delivery system 14 (without bleed valve 30) was assembled and tested. The gas cylinder 22 was pressurized to 100 psig with oxygen. The pressurized prototype weighed approximately 300 grams. The regulator 26 was set to maintain an output pressure of 5 psig. The pressurized prototype was placed into operation, and the flow rate of oxygen output from the flexible tubing (or cannula) 28 was monitored. The prototype delivered a substantially constant rate of oxygen flow for approximately 20 hours. During this period, the oxygen flow rate remained steady at approximately 3.3 mL/hr. In view of these results, the gas cylinder may be charged with therapy gas to a higher pressure (e.g., up to 500 psig with the presently disclosed apparatus, or up to approximately 1000 psig with a suitable regulator, hose and fittings) to achieve continuous oxygen delivery at a similar rate for up to seven days. Alternatively, a gas cylinder with greater storage capacity may be used at a lower therapy gas output pressure.

Accordingly, the portable therapy gas storage and delivery assembly 14 may be selectively adjusted to supply therapy gas continuously (at a constant or variable rate of flow) for up to 7-10 days at a desired regulator output pressure and/or flow rate. For example, preliminary test results indicate that a minimum pressure of 5 psig may be required to effectively kill bacteria in a chronic wound that is undergoing continuous transdermal gas therapy. In one therapeutic configuration, the regulator may be set to deliver a therapy gas at a substantially constant rate of flow (e.g., 3.3 mL/hr) while maintaining a steady output pressure of at least 5 psig for approximately 7 days. The therapy gas may be a mixture of medical grade air and nitric oxide. The nitric oxide may be 1% (by mass) of the gas mixture when measured at standard temperature and pressure. The regulator output pressure may be set to maintain a steady output pressure of approximately 5 psig. Nevertheless, the therapeutic configuration may be modified, adjusted, or optimized based on one or more considerations or factors that may include, without limitation, the composition of the therapy gas, the altitude of the treatment location, the clinical objective of the treatment, and the efficacy of trial therapies studied in a laboratory and/or clinical setting.

Referring to FIG. 2, the therapy gas from the gas storage and delivery assembly 14 may be incorporated with a disposable, sterile wound dressing 36. The wound dressing 36 may include a therapy gas discharge structure, as well as a mechanical apparatus for securing the therapy gas discharge structure proximate to the wound. The therapy gas discharge structure may be fluidly connected to the flexible tubing (or cannula) 28 from the pressure regulator 26. The mechanical apparatus for securing the therapy gas discharge structure proximate the wound may include an inflatable structure 38 and a pump 40 for inflating the structure.

The mechanical pump 40 may be a miniature diaphragm pump which may be driven by a DC motor. At standard conditions, the pump may operate over a pressure range of approximately 0 kPa to 165 kPa and a vacuum range of approximately 0 mmHg to 500 mmHg absolute. The maximum unrestricted flow of the pump may be approximately 2.5 liters per minute (LPM). Operation of the mechanical pump may be controlled via pulse width modulation of the DC motor. One commercially available pump which may be suitable for this application is a 2.5 LPM CTS Micro Diaphragm Pump manufactured by Parker Hannifin Corporation. Although a diaphragm pump may be used to inflate the wound dressing 36, any suitable fluid delivery system (e.g., a spring loaded piston) may be used to pressurize the inflatable structure 38 provided that the inflatable structure securely positions the therapy gas discharge structure (e.g., the aperture) proximate the wound and creates a gas tight seal between the edge of the dimple and the abutting surface (e.g., tissue, gasket material, sealant).

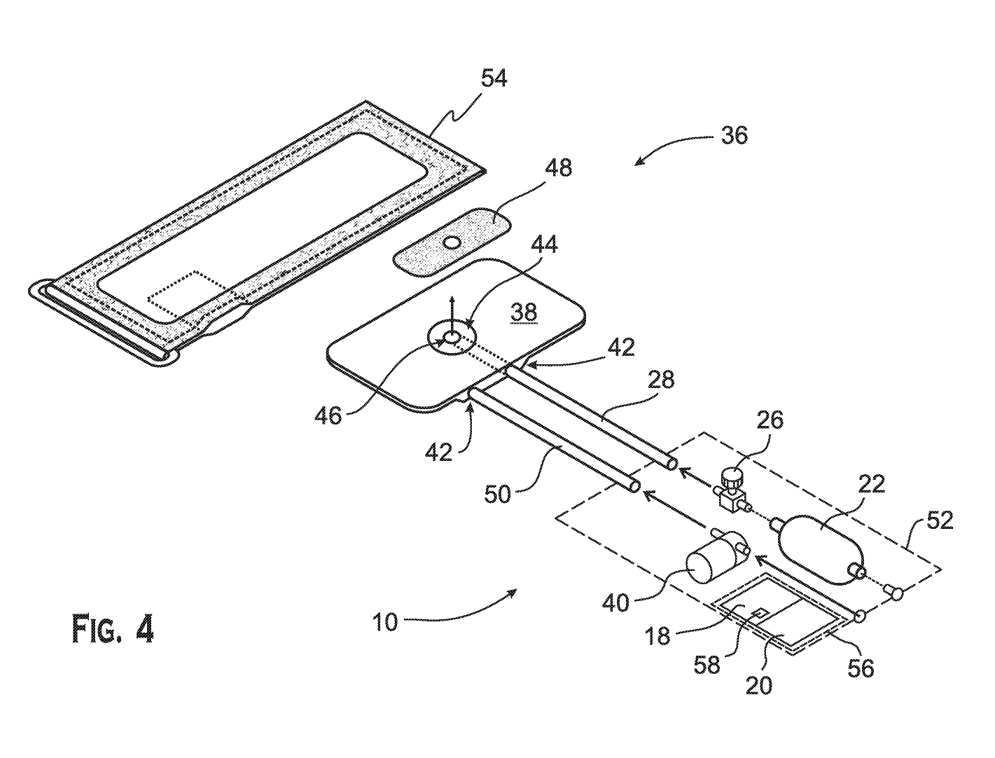

Referring to FIG. 4, in an exemplary embodiment the wound dressing 36 may include the discharge end of the flexible tubing 28 from the regulator 26 and therapy gas storage tank assembly. The flexible tubing 28 may pass through the inflatable structure 38 to deliver the therapy gas to the wound. Penetrations 42 in the inflatable structure 38 that provide the flexible tubing portable therapy gas storage and delivery assembly 14 with access to the wound may be sealed. The flexible tubing 28 may be positioned directly near the wound or connected to a sealed pocket or dimple 44 within the inflatable structure, which may include one or more openings (or apertures) 46 for delivering discharge therapy gas to the wound.

The wound dressing 36 further may include a gasket 48 and sealing materials which may be placed in direct contact with skin adjacent to the wound to create a sealed chamber about the wound. For example, a gasket material 48 formed from silicone may be cut by a caregiver to fit the wound site. The silicone material may include a metal foil coating.

Additionally, the gasket material 48 may include an adhesive surface to seal the gasket to the patient's skin in an effort to prevent leaks from developing between the wound dressing and the patient's skin. Additional adhesives or sealants may be used to isolate the cavity. The inflatable structure 38 and any gasket materials and sealants used to create the sealed cavity about the wound may be compatible with skin contact. The sealed cavity then may be filled with therapy gas to provide an atmosphere of therapy gas proximate the wound.

The inflatable structure 38 may be shaped for a particular anatomy to help secure the dimple 44 about the wound. In one embodiment, the inflatable structure 38 may be rectangular in shape, and the therapy gas discharge site 46 may be located in the middle of the rectangular shape. The inflatable structure 38 may be draped over the wound and inflated to create a sealed cavity about the wound. Using an inflatable structure (e.g., a bladder) 38 to create a sealed cavity about the wound may distribute compressive forces applied to the patient by the dressing 36. This may prevent concentrated forces from being applied to the patient. The inflatable structure 38 may be connected to a mechanical pump 40 via a conduit 50, such as flexible tubing 26. The pump 40 may be a hand pump, a spring loaded piston, an electrically operated mechanical pump, or other suitable pump provided the pump supplies sufficient fluid flow rates and discharge pressure to expand the inflatable structure 38 and achieve a sealed cavity about the wound. Components of the apparatus 10 may be contained in a housing 52 which may be secured to the wound dressing.

The wound dressing 36 may include a cuff 54 or similar garment to secure the inflatable structure 38 to the patient. In one embodiment, the cuff 54 or other garment wraps around the inflatable structure 38 to fix the inflatable structure with respect to the wound. The cuff may be secured, for example, by one or more straps or a plurality of hook and latch structures (e.g., Velcro strips).

The treatment system 10 further may include a control circuit 56 for regulating operation of the gas storage and delivery apparatus 14 and the pressurized dressing delivery apparatus 16. The treatment system 10 may include a power supply 21 for supplying electricity to the air pump 40 and the control circuit 56. The control circuit may monitor process variables from the gas storage and delivery apparatus 14, the pressurized dressing and delivery apparatus 16, the user interface 20, and the power supply 21. The control circuit 56 may include a microprocessor and memory, an application specific integrated circuit (ASIC), and/or a microcontroller 58 that is in electrical communication with other components and sensors in order to regulate operation of the treatment system 10. For example, the control circuit 56 may monitor and analyze the therapy gas flow rate and discharge pressure from the therapy gas storage tank to regulate operation of the mechanical pump.

Additionally, the control circuit 56 may monitor and analyze the therapy gas delivery pressure and flow rate between the pressure regulator 26 and the therapy gas discharge structure 46. Also, the control circuit 56 may monitor and analyze the fluid pressure in the inflatable structure 38, as well as operating parameters of the air pump 40. The control circuit 56 further may include a user interface 20 for powering the system on and off, as well as announcing the operational state of each process apparatus 14, 16. For example, the user interface 20 may include a power button and a display 20.

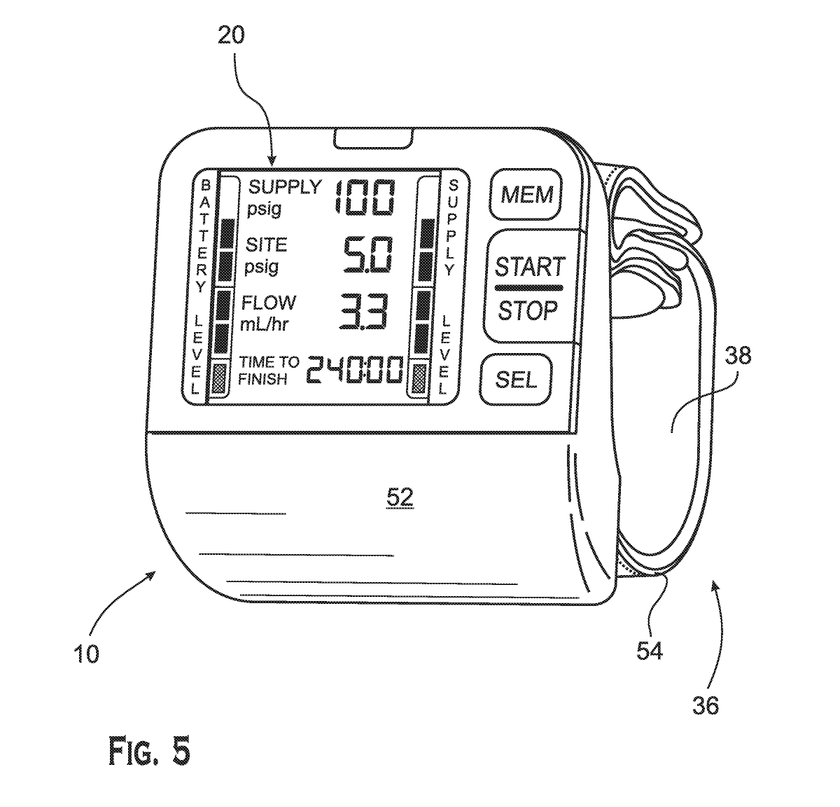

Referring to FIG. 5, the display 20 may provide a graphical user interface for reporting the state of process variables that may be monitored by the control circuit 56. For example, the graphical user interface may report the state of one or more of the following process variables: air pump condition, gas pump condition, bladder pressure, gas tank pressure, gas flow rate, treatment time, purge control, and battery charge.

Based on sensor data signals for monitored process variables, the control circuit 56 may regulate operation of the system 10. For example, the control circuit 56 may monitor the pressure of the inflatable structure 38 and control operation of the air pump 40 to maintain a desired pressure. Similarly, the control circuit 56 may monitor the pressure and discharge rate of therapy gas exiting the storage tank 22 and the pressure and discharge rate of therapy gas exiting the pressure regulator 26 to track and assess system performance. The control circuit 56 may be configured to generate audible or visual alarm based on high and low pressure measurements, differential pressure measurements, and therapy gas flow rates.

The control circuit 56 may be configured to implement prescribed functions. For example, without limitation, the control circuit 56 may be configured to report on elapsed treatment time, perform a purge of therapy gas from the wound dressing, and monitor power supply levels. Other functionality may be programmed or configured into the control circuit components as well. For example, a microcontroller may instruct the system to shut off gas flow from the storage tank 22 in the event that therapy gas is leaking from the cavity 44 at an unacceptable rate. In another example, a microcontroller may be programmed to prevent over-inflation of the inflatable structure 38.

The control circuit 56 further may be implemented to monitor sensor data which report one or more vital signs of the patient undergoing treatment with the device. For example, the inflatable structure 38 may include sensors for measuring skin temperature, pulse rate, respiration rate, or blood pressure. The control circuit 56 may monitor one or more of these vital signs of the patient to provide process control feedback, including safety alarms to prevent harm or excessive discomfort to the patient. For example, the control circuit 56 may instruct the system 10 to turn off or reduce the pressure of the inflatable bladder 38 should a vital sign measurement (e.g., the pulse rate or blood pressure) depart from a target level or range of values.

Referring to FIG. 5, the gas storage and delivery apparatus 14 may be contained in a housing 52, along with a power supply, control system and user interface 20. By contrast, the cuff 54 and inflatable structure 38 may be attached to an exterior portion of the housing 52. The housing 52 may be separable from the cuff 54 and the inflatable structure 38. Moreover, the cuff 54 and inflatable structure 38 may be modular such that a used cuff and wound dressing assembly may be disconnected from the housing for disposal and then replaced with a new sterile cuff and wound dressing assembly. In this manner, the housing 52 may be reusable, but the cuff 54 and inflatable structure 38 may be disposable.

The size and configuration of the housing 52 may depend on the nature or location of the wound being treated. Accordingly, the size and configuration of the housing 52 may be determined, in part, by the size and configuration of the gas storage tank 22 and power supply 21. In the disclosed embodiment, the housing 52, cuff 54 and inflatable structure 38 are shown in a mobile device, which may be similar to commercially available wrist-fitting blood pressure monitoring systems.

In use, the gas storage tank 22 may be filled from a high pressure supply of medical grade gas. A shut-off valve 24 on the gas storage tank 22 may be closed, and the regulator 26 of the gas delivery device may be disconnected from the shutoff valve 24. The shut-off valve 24 on the gas storage tank may then be connected to the source of medical grade gas 11. For example, the shut-off valve on the gas storage tank may be connected to a gas cylinder containing USP grade nitric oxide 1% in nitrogen with a supply pressure of approximately 100 psig. The shut-off valve 24 may then be opened to allow the gas storage tank 22 to fill with the therapeutic gas mixture. After the gas storage tank 22 is charged, the shut off valve 24 is closed, the source of medical gas 11 is disconnected from the shut-off valve, and the regulator 26 is reconnected to the shut-off valve. The regulator 26 then may be adjusted to deliver gas at a target delivery pressure. For example, the regulator 26 may be set to reduce the tank discharge pressure from approximately 100 psig to substantially equal to or greater than 5 psig.

The wound area may be prepared by cleaning the wound area and securing a silicone gasket about the wound. The gasket 48 may include an adhesive material on the side facing the patient's skin in order to prevent fugitive emissions of therapy gas from escaping between the skin and the inflatable structure 38. The inflatable structure then may be placed over the wound and gasket 18. Additional sealant may be placed between the silicone gasket 18 and the inflatable structure 38 to prevent fugitive emissions of therapy gas from breaking through the interface of the silicone gasket and the inflatable structure. The cuff then may be placed around the inflatable structure and synched to hold the wound dressing firmly against the patient.

The shut-off valve 24 may be opened to release therapy gas to the wound dressing assembly 36. The therapy gas may fill the cavity formed by the dimple 44 in the inflatable structure 38 and the silicone gasket 18. The control circuit 56 may be powered on, and a microcontroller (or equivalent device(s)) may activate the air pump 40 to inflate the inflatable structure 38. Periodically, as active agent(s) in the therapy gas are consumed and exhausted, the atmosphere in the cavity may be purged to allow fresh therapy gas to recharge the chamber with active agent(s). This may be done manually by a user or automatically under the regulation of the control unit. Purging the cavity may include: closing the shut-off valve 24; opening the bleed valve (or purge valve) 30; allowing the pressure in the cavity to subside; and then opening the shut-off valve 24. Residual active agents in the purge gas (e.g., NO) may be removed by a suitable treatment process. After the exhausted therapy gas is purged and fresh therapy gas is supplied to the cavity, the bleed valve 30 may be closed to seal the apparatus 14 and start another treatment cycle. After treatment has been completed, the control circuit may be powered off, and the disposable components removed from the system 10 and discarded.

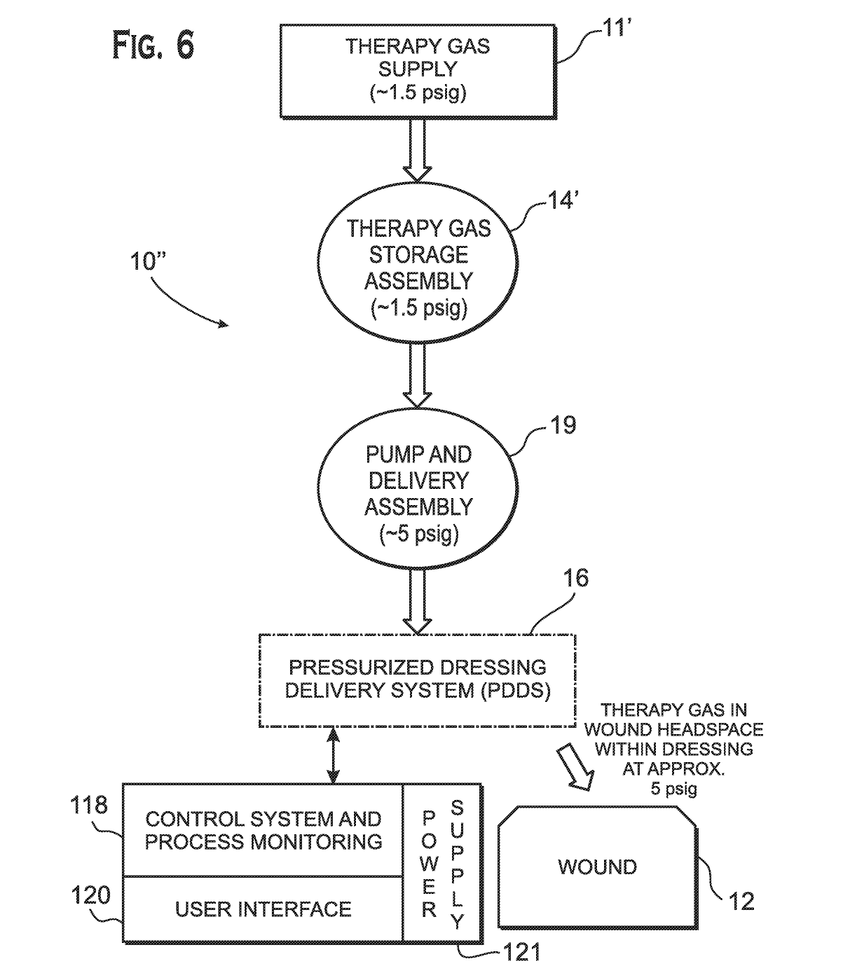

FIG. 6 presents a block diagram of another apparatus 10″ for delivering therapeutic gas (therapy gas) to a wound 12. The apparatus 10″ includes a gas storage and delivery assembly 14′ for supplying the pump and delivery assembly 19 with therapy gas for treating a wound, a pressurized dressing 16 for delivering therapy gas to the wound 12, and a control system 18 which may include a user interface 20 and power supply 21 for regulating operation of the system 10″.

The gas storage assembly 14″ may be configured and designed to store therapy gas at pressures lower than the delivery pressure of the therapy gas at the wound site. For example, the gas storage assembly 14″ may be designed to store therapy gas at approximately 1.5 psig. The gas storage assembly 14″ may be designed to supply the mechanical pump and delivery assembly 19 with therapy gas for a target time period. The target time period may correspond with a desired treatment cycle. For example, the therapy gas storage assembly 14″ may be designed to supply the pump and delivery assembly 19 with therapy gas for approximately 7 days.

The pump and delivery assembly 19 may include a mechanical pump 40′ which is fluidly connected to the therapy gas storage assembly 14″. The mechanical pump 40′ may pressurize the therapy gas from the storage assembly to provide a supply of therapy gas to the dressing 36 and wound headspace (at a constant or variable rate of flow) at a higher pressure than the pressure of the therapy gas in the storage assembly. For example, the mechanical pump and delivery system 19 may pressurize the therapy gas from the storage assembly 14′ to supply therapy gas to the wound headspace within the dressing at approximately 5 psig.

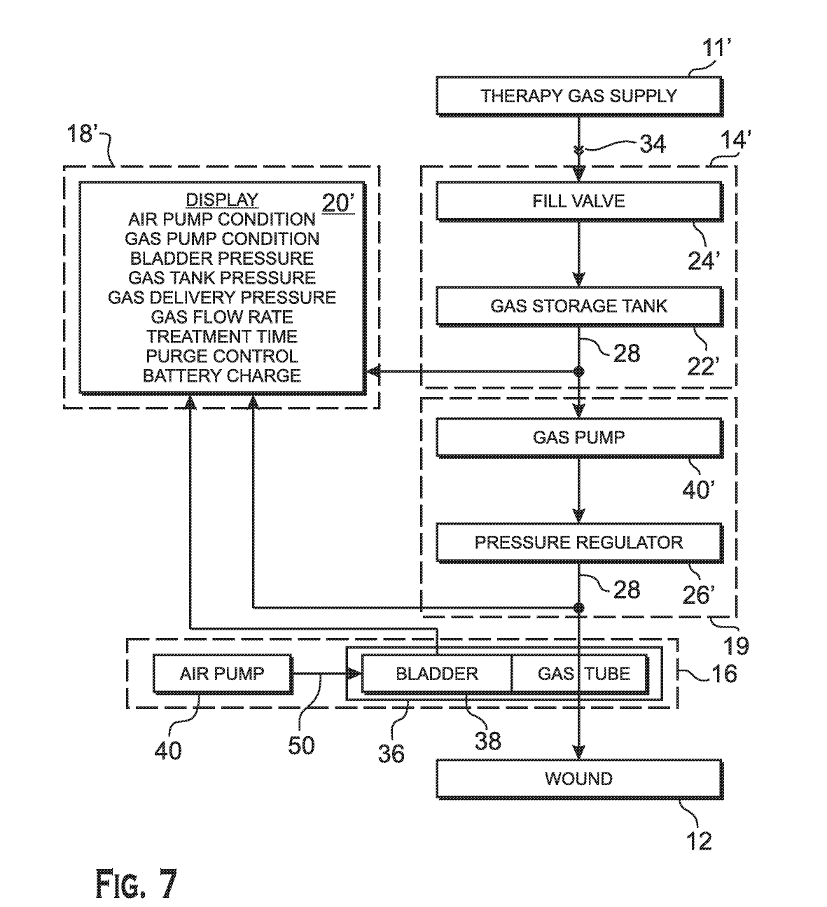

Referring to FIG. 7, the therapy gas storage and delivery assembly 14′ may include a gas storage tank 22′, fittings (e.g., fill valve 24′ and pressure regulator 26′), and flexible tubing 28 which supply the apparatus with medical grade therapy gas. The tank 22′ may be designed to store and dispense a single charge of therapy gas. The tank 22″ may be separable from the therapy gas storage assembly 14′.

The gas storage tank may be formed from a suitable thermoset material, thermoplastic material, a polymer composition, a fiberglass material or other material. For example, the gas storage tank may be formed from a metal or a metal alloy (e.g., steel, aluminum, or stainless steel). The material further may include a glass fiber or synthetic fiber reinforcing material (e.g., nylon or Kevlar). Additionally, the gas storage tank may be constructed from a combination of materials. For example, the tank may be constructed with a metal liner with full composite overwrap (e.g., aluminum, with a carbon fiber composite). In another example, the tank may be constructed from a polymer (e.g., high-density polyethylene or HDPE) liner with carbon fiber or hybrid carbon/glass fiber composite materials.

The mechanical pump 40′ may deliver therapy gas from the gas storage tank 22′ to a pressure regulator 26′. The pressure regulator 26′ may reduce and regulate the pressure of the therapy gas supplied by the mechanical pump 40′ to deliver and maintain a steady output pressure within the dressing 36 at the wound. For example, the mechanical pump may deliver therapy gas at a substantially constant rate of flow (e.g., 3.3 mL/hr) and at a pressure in excess of 6 psig, and the regulator may reduce or regulate the pressure of the therapy gas supplied by the mechanical pump to maintain a steady output pressure of approximately 5 psig.

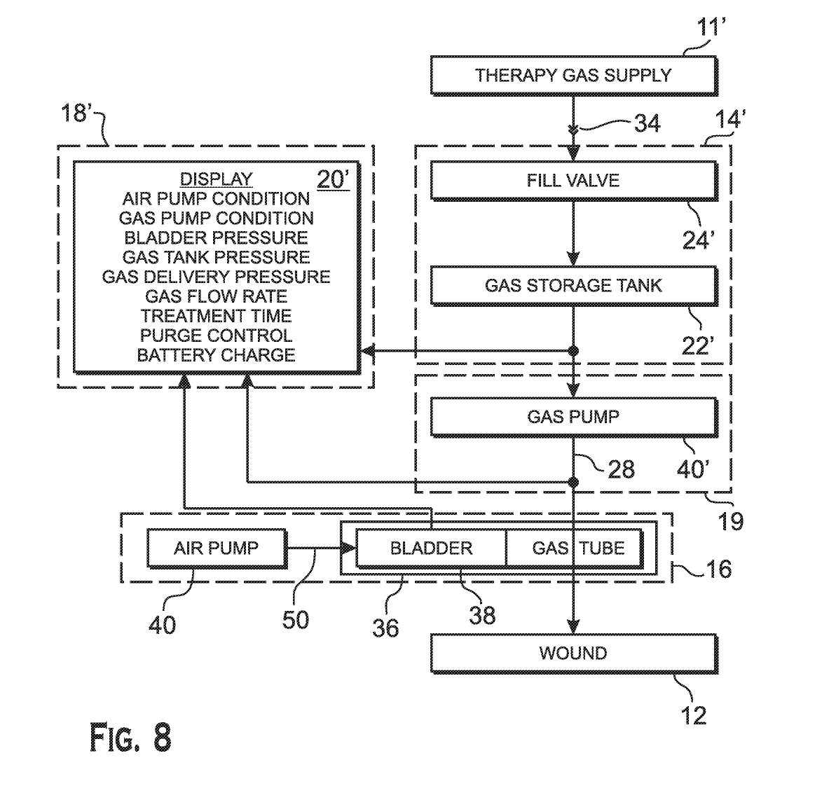

As shown in FIG. 8, the mechanical pump 40′ may supply therapy gas to the wound headspace within the dressing without a pressure regulator. In this embodiment, the control circuit 56 may regulate operation of the mechanical pump 40′ to achieve the desired pressure within the dressing 36 at the wound headspace. In this configuration, the pressure profile of the therapy gas may range from 0 psig to a set point for the maximum desired pressure within the dressing at the wound headspace.

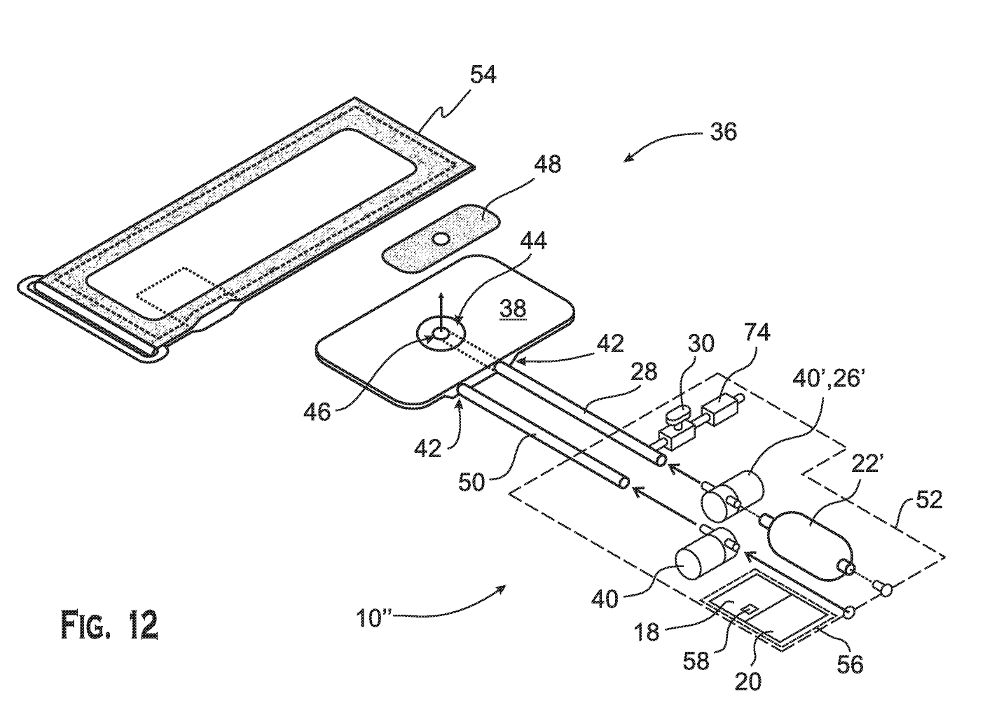

As shown in FIG. 11, the wound dressing 36′ may include an inflatable structure 38 that is shaped as a tubular member that may be configured and dimensioned to surround a limb, torso, or extremity 70. The tubular member may be used to create and secure a sealed cavity about a wound site. The wound dressing may include the discharge end of the flexible tubing 28 from the regulator 26 and therapy gas storage tank assembly 14. The flexible tubing 28 may pass through the inflatable structure 38 to deliver the therapy gas to the wound. Penetrations 42 in the inflatable structure that provide the flexible tubing with access to the wound may be sealed. The flexible tubing 28 may be positioned directly near the wound or connected to a pocket (or dimple) 44 in inflatable structure 38, which may include one or more openings (or apertures) 42 for delivering therapy gas to the wound. The tubular member may be formed from a soft, flexible, and gas tight material. For example, the tubular member may be formed from an elastomeric material. The tubular member may be covered with another fabric 72. The fabric cover 72 may be a moisture wicking material. The fabric cover may be impregnated or include one or more coatings of antibacterial agents, therapeutic agents, deodorants, or absorbents. The fabric may be formed from natural or synthetic fibers.

The wound dressing 36 further may include a gasket 48 and sealing materials which may be placed in direct contact with skin adjacent to the wound to create a sealed chamber about the wound. For example, a gasket material 48 formed from silicone may be cut by a caregiver to fit the wound site. The silicone material may include a metal foil coating. Additionally, the gasket material may include an adhesive surface to seal the gasket to the patient's skin in an effort to prevent leaks from developing between the wound dressing and the patient's skin. Additional adhesives or sealants may be used to isolate the chamber. Preferably, the inflatable device 38 and any gasket materials 48 and sealants used to create the sealed cavity about the wound may be compatible with skin contact. The sealed cavity may then be filled with therapy gas to topically treat the wound.

The inflatable structure 38 may be shaped for a particular anatomy to help secure the dimple about the wound. In one embodiment, the inflatable structure 38 may be generally rectangular in shape, and the therapy gas discharge site 44 may be located in the middle of the inflatable structure. The inflatable structure may be draped over the wound and inflated to create a tightly sealed chamber. Using an inflatable structure (e.g., a bladder) to create a tightly sealed chamber may distribute compressive forces applied to the patient by the dressing. The inflatable structure may be configured and dimensioned to surround a particular anatomy (e.g., a toe, a foot, an arm, a leg, a torso, and such.) The inflatable structure may be adapted for use in treating dogs, horses or other animals.

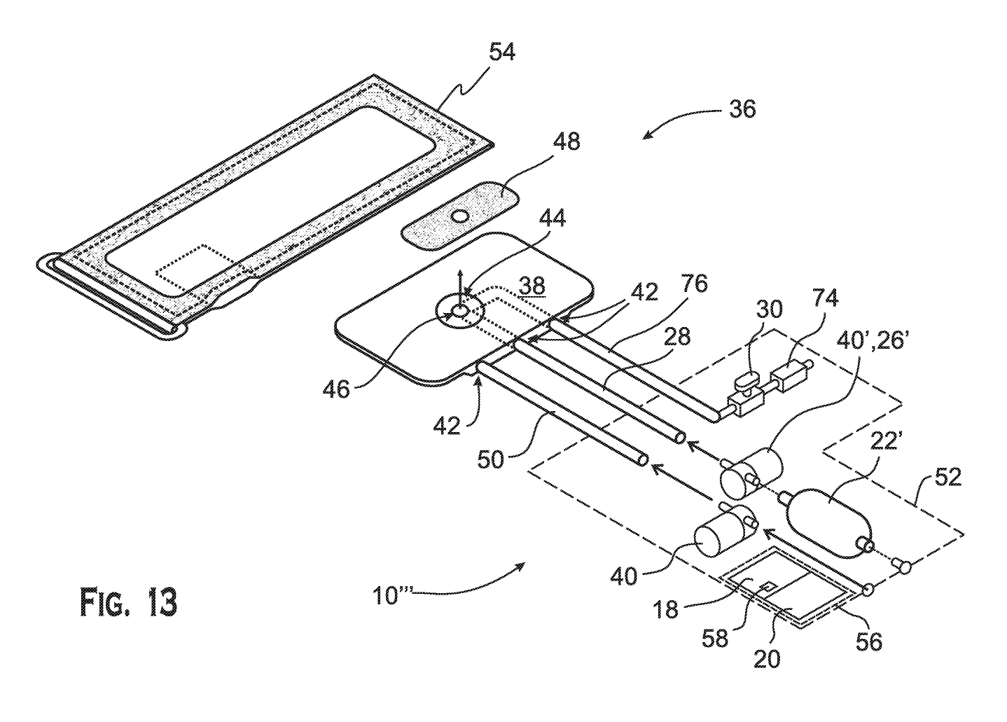

FIG. 12 shows another embodiment of a mobile dressing 36 for therapeutically delivering pressurized gas to a wound. The mobile dressing 36 may include an inflatable structure 38 with one or more openings (or apertures) 46 for supplying therapy gas to a wound, a lumen 28 fluidly connected to the one or more openings 46 in the inflatable structure (or bladder) 38, and another lumen 50 for supplying compressed air to inflate the inflatable structure 38. The mobile dressing may include a fabric wrap 54 with Velcro fastener strips which may be used to securely position the bladder at the treatment site. The pressure dressing 36 is substantially the same as the mobile dressing presented in FIG. 4, except that a low pressure storage tank 22′ and a mechanical pump 40′ may be used to deliver therapy gas to the lumen 28 which is fluidly connected to the one or more openings 42 in the inflatable structure 38. For instance, the therapy gas may be stored at approximately 1.5 psig and delivered to the one or more openings at approximately 5 psig. Moreover, the therapy gas supply lumen may include a purge valve 30 and a capture device 74. The purge valve 30 may be normally closed during wound care. The purge valve, however, may be opened to purge the headspace of the wound. Purged gases may be captured in the capture device 74. The capture device 74 may include a catalyst that renders the purged gases harmless. The capture device may include an adsorbent or molecular sieve that safely stores the purged gases. The catalyst, adsorbent, or molecular sieve may be adapted for a particular therapy or carrier gas. For example, a platinum-based catalyst may be used to oxidize or reduce gaseous compounds to less objectionable compounds so that they can be released to the atmosphere.

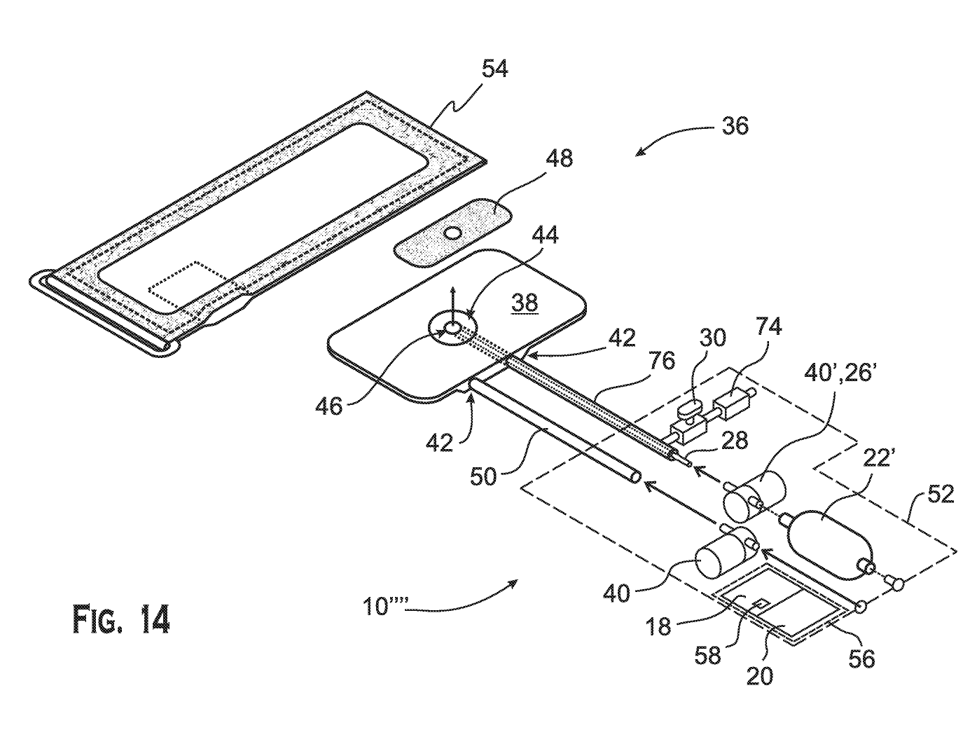

FIG. 13 shows another embodiment of a mobile system 10″′ for delivering pressurized gas to a wound. In this embodiment, a third lumen 76 is fluidly connected to the one or more openings 42 in the inflatable structure 38. The third lumen 76 may be fluidly connected to the purge valve 74 and the capture device. In this configuration, the purge valve 30 may be opened and therapy gas may be used to flush the first lumen 28, the dimple 44 cavity, and the third lumen 76 with therapy gas. Additionally, the intake of the mechanical pump 40′ which delivers the therapy gas to the first lumen 28 may be selectively connected to a vent (not shown) to provide a supply of ambient air for purging gases from the wound headspace.

FIG. 14 shows yet another embodiment of system for therapeutically delivering pressurized gas to a wound. In this embodiment, the first lumen 28 and the third lumen 76 of FIG. 13 may be replaced by a multiple-lumen tubing. For example, the inner lumen 28′ may functionally correspond to the first lumen 28 (FIG. 13) and may deliver pressurized therapy gas to the one or more openings 46 in the inflatable structure 38. The outer lumen 76′ may functionally correspond to the third lumen 76 (FIG. 13) and may transport gases from the one or more openings 46 to the purge valve 30 and capture device 74.

Various gases may be administered to a wound in an intermittent, sequential, or combined configuration to address a particular therapeutic goal. For example, nitric oxide may be applied to kill bacteria; and then oxygen may be applied to enhance growth factors and granulation. In another example, ozone may be applied to kill bacteria, followed by the application of oxygen to enhance growth factors and granulation. In yet another example, nitric oxide may be applied to kill bacteria and then nitrogen or carbon dioxide may be administered to enhance angiogenesis.

While it has been illustrated and described what at present are considered to be embodiments of the present invention, it will be understood by those skilled in the art that various changes and modifications may be made, and equivalents may be substituted for elements thereof without departing from the true scope of the invention. For example, a different pump may be used to inflate the inflatable structure or to pressurize a low pressure supply of therapy gas. In another example, the fabric cover may be disposable and thus the cuff may be reusable with one or more replacement fabric covers. Additionally, features and/or elements from any embodiment may be used singly or in combination with other embodiments. Therefore, it is intended that this invention not be limited to the particular embodiments disclosed herein, but that it have the full scope defined by the language of the following claims, and equivalents thereof.

What is claimed is:

1. A mobile dressing for therapeutically delivering pressurized therapy gas to a wound comprising:
an inflatable structure for creating a sealed cavity around a wound which comprises
a flexible container for receiving pressurized gas, the flexible container comprises
an interior chamber for containing pressurized gas, and
an opening in the interior chamber for receiving pressurized gas,
a wound-facing surface which comprises
a dimple, and
an aperture within the dimple,
a first lumen fluidly connected to the aperture for delivering pressurized therapy gas to the aperture, and
a second lumen fluidly connected to the opening for delivering pressurized gas to the interior chamber;
a first pump which comprises
a first intake port fluidly connected to a first reservoir of therapy gas at a first pressure, and
a first discharge port fluidly connected to the first lumen for supplying therapy gas from the first reservoir at a second pressure that is greater than the first pressure;
a second pump which comprises
a second intake port fluidly connected to a second reservoir of gas at a third pressure, and
a second discharge port fluidly connected to the second lumen for supplying pressurized gas from the second reservoir at a fourth pressure that is greater than the third pressure;
the mobile dressing having a plurality of operable configurations which comprise
a first operable configuration such that the interior chamber contains a first volume of gas from the second reservoir at the third pressure, and
a second operable configuration such that the interior chamber contains a second volume of gas from the second reservoir at the fourth pressure, and the first discharge port supplies therapy gas from the first reservoir to the aperture at the second pressure.

2. The mobile dressing of claim 1, wherein the second reservoir is the atmosphere.

3. The mobile dressing of claim 2, wherein the pressurized gas is air.

4. The mobile dressing of claim 1, wherein the first reservoir of therapy gas is a tank.

5. The mobile dressing of claim 4, wherein the tank is designed to store therapy gas at a pressure substantially equal to or less than 5 psig.

6. The mobile dressing of claim 5, wherein the tank is a refillable gas storage device.

7. The mobile dressing of claim 5, wherein the tank is configured to store and dispense a single charge of therapy gas.

8. The mobile dressing of claim 4, further comprising a purge valve fluidly connected to the first lumen.

9. The mobile dressing of claim 8, further comprising a capture device fluidly connected to the purge valve.

10. The mobile dressing of claim 9, wherein the capture device comprises a catalyst that interacts with purged therapy gas to render purged therapy gas harmless.

11. The mobile dressing of claim 10, wherein the catalyst is a platinum-based catalyst.

12. The mobile dressing of claim 9, wherein the capture device comprises an adsorbent that safely stores purged therapy gas.

13. The mobile dressing of claim 9, wherein the capture device comprises a molecular sieve that safely stores purged therapy gas.

14. The mobile dressing of claim 9, further comprising a third lumen fluidly connected to the aperture, wherein the purge valve is connected to the third lumen.

15. The mobile dressing of claim 14, wherein the first lumen and the third lumen are two lumen in a multi-lumen tubing.

16. The mobile dressing of claim 1, wherein the first pressure is approximately 1.5 psig and the second pressure is approximately 5 psig.

17. The mobile dressing of claim 1, further comprising a pressure regulator, the pressure regulator being fluidly connected to the first discharge port and the first lumen such that in the second operable configuration the first discharge port supplies therapy gas to the pressure regulator at a pressure substantially equal to or greater than 6 psig and the pressure regulator supplies therapy gas to the first lumen at a pressure of approximately 5 psig.

18. The mobile dressing of claim 1, wherein the first reservoir of gas is stored in a pressure cylinder capable of supplying stored gas continuously for 10 days at a discharge pressure ranging from approximately 1.1 psig to approximately 1.5 psig.

19. The mobile device of claim 1, wherein the therapy gas is selected from a group of gases consisting of oxygen, ozone, chlorine dioxide, nitrogen, nitric oxide, medical grade air, or a mixture thereof.

20. The mobile device of claim 1, wherein the therapy gas is a mixture of 1% nitric oxide and 99% nitrogen.
